(19) United States
(12) Patent Application Publication
(10) Pub. No.: US 2025/0200951 A1
(43) Pub. Date: Jun. 19, 2025

(54) AUTOMATED TEST OF EMBODIED COGNITION

(71) Applicants: UNITED STATES GOVERNMENT AS REPRESENTED BY THE DEPARTMENT OF VETERANS AFFAIRS, Washington, DC (US); BOARD OF REGENTS, THE UNIVERSITY OF TEXAS SYSTEM, Austin, TX (US); YALE UNIVERSITY, New Haven, CT (US)

(72) Inventors: Morris Bell, New Haven, CT (US); Fillia Makedon, Arlington, TX (US); Vassilis Athitsos, Arlington, TX (US)

(21) Appl. No.: 18/843,593

(22) PCT Filed: Mar. 3, 2023
(86) PCT No.: PCT/US2023/014495

§ 371 Date: Sep. 3, 2024

Related U.S. Application Data
(60) Provisional application No. 63/316,747, filed on Mar. 4, 2022.

(51) Int. Cl. G06V 10/776; G06T 7/00; G06T 7/246; G06V 10/774; G06V 10/82; G06V 40/20

(52) U.S. Cl. CPC G06V 10/776; G06T 7/0016; G06T 7/251; G06V 10/774; G06V 10/82; G06V 40/23; G06T 2207/20081; G06T 2207/20084; G06T 2207/30196

(57) ABSTRACT

Methods, systems, and apparatuses for predicting one or more neurological assessments or one or more cognitive scores associated with the observed movements of a subject. Motion data associated with movements of one or more subjects may be used to train a predictive model. The predictive model may be trained to output a prediction indicative of one or more neurological assessments or one or more cognitive scores of a subject.

### CROSS REFERENCE TO RELATED PATENT APPLICATION

This Application claims priority to U.S. Provisional Application No. 63/316,747, filed Mar. 4, 2022, which is herein incorporated by reference in its entirety.

### BACKGROUND

The field of Embodied Cognition has emerged from neuroscience understandings of cognitive development and neuroplasticity that make it clear that cognition develops along with and by way of physical movement, that cognitive health is maintained with physical activity and that cognitive decline increases with inactivity. The brain is organized by integrated circuits so that even executive functions such as working memory, self-regulation and planning associated with the pre-frontal cortex are now understood to be part of complex systems that include cerebellar regions usually associate with movement and balance. While neuropsychological assessment methods are anchored in a strong foundation of cognitive science, its procedures are based on disembodied and localizationist-connectionist approaches. This classic model of perception-cognition-action ignores the sensory-motor system and its bodily and emotional experience. Increasingly, research shows how action and emotions influence perception and higher-level cognition, just as higher-level cognition can alter actions and emotions. There is currently no assessment system for Embodied Cognition. There are some occupational therapy and neurological assessments that are related to embodied cognition. However, they are not cognitively demanding tasks and do not relate to executive functioning. While digital technologies are currently available to evaluate physical movements such as gait analysis using motion sensors, there has been no use of motion capture technology and machine learning algorithms to analyze executive functions in action.

One of the biggest issues facing the use of machine learning is the lack of availability of large, annotated datasets. The annotation of data is not only expensive and time consuming but also highly dependent on the availability of expert observers. The limited amount of training data can inhibit the performance of supervised machine learning algorithms which often need very large quantities of data on which to train to avoid overfitting. So far, much effort has been directed at extracting as much information as possible from what data is available. One area in particular that suffers from lack of large, annotated datasets is analysis of movement data, such as motion data. The ability to analyze movements of a subject to predict neurological assessments is critical to patient care.

### SUMMARY

It is to be understood that both the following general description and the following detailed description are exemplary and explanatory only and are not restrictive. Methods, systems, and apparatus for improved capability to determine and utilize movement data sets for training machine learning applications to make predictions, including predicting patient neurological assessments, are described.

In an embodiment, disclosed are methods comprising determining motion data associated with a plurality of movements, wherein the plurality of movements include one or more series of movements, wherein each series of movements of the plurality of movements is labeled according to a predefined feature of a plurality of predefined features, determining, based on the motion data, a plurality of features for a predictive model, training, based on a first portion of the motion data, the predictive model according to the plurality of features, testing, based on a second portion of the motion data, the predictive model, and outputting, based on the testing, the predictive model.

In an embodiment, disclosed are methods comprising receiving baseline feature data associated with a plurality of movements of a subject, wherein the plurality of movements are determined from a plurality of observed movements, providing, to a predictive model, the baseline feature data, and determining, based on the predictive model, a neurological assessment of the subject.

In an embodiment, disclosed are methods comprising determining baseline feature data associated with a plurality of movements of a subject, wherein the plurality of movements include one or more series of movements, wherein each series of movements of the plurality of movements is labeled according to a predefined feature of a plurality of predefined features.

In an embodiment, disclosed are methods comprising wherein the baseline feature data associated with a plurality of movements of a subject comprises an embodied cognitive task that includes a cognitive component and a physical component, wherein the subjects performance is assessed on each of the cognitive component and the physical component, wherein the embodied cognitive task is presented to the subject via a display component.

In an embodiment, disclosed are methods comprising wherein the embodied cognitive task is a video game.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of the present description serve to explain the principles of the methods and systems described herein:

FIG. 1 shows an example system;

FIG. 2 shows an example model architecture;

FIG. 3 shows an example joint representation of a subject;

FIG. 4 shows a flowchart of an example method;

FIG. 5 shows an example machine learning system;

FIG. 6 shows a flowchart of an example machine learning method;

FIGS. 7A-7D show example prediction results;

FIG. 8 shows a block diagram of an example system;

FIG. 9 shows a flowchart of an example method;

FIG. 10 shows a flowchart of an example method;

FIG. 11 shows an example computing system for presenting embodied cognitive tasks to a subject;

FIG. 12 shows an example computing system for presenting embodied cognitive tasks to a subject; and

FIGS. 13A-13B show example scenarios of a subject performing embodied cognitive tasks.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. Similarly, when values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal configuration. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, the methods and systems may take the form of a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

Methods and systems are described for generating a machine learning classifier for the prediction of one or more neurological assessments associated with a plurality of movements. Machine learning (ML) is a subfield of computer science that gives computers the ability to learn without being explicitly programmed. Machine learning platforms include, but are not limited to, naïve Bayes classifiers, support vector machines, decision trees, neural networks, and the like. In an example, baseline feature data may be obtained for a plurality of movements of a subject. The baseline feature data may be analyzed to determine one or more neurological assessments. The one or more neurological assessments may comprise a likelihood of a deficiency of a subject's motor function, balancing, reflex movement, sensory function, coordination, or gait. In an example, baseline feature data and/or curated feature data from one or more other studies may be analyzed to determine one or more predictive neurological assessments. In an example, the baseline feature data may be analyzed to determine one or more cognitive scores for determining the one or more neurological assessments. The one or more cognitive scores may comprise an action score, a rhythm score, and a function score.

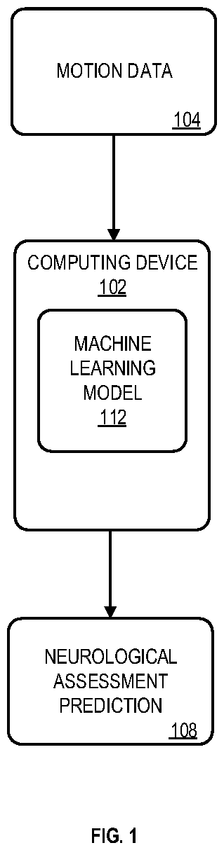

FIG. 1 shows a system 100 that may be configured to determine a one or more neurological assessments of a subject. The one or more neurological assessments may comprise a likelihood of one or more of a deficiency of a subject's motor function, balancing, reflex movement, sensory function, coordination, or gait. As an example, the one or more neurological assessments may be determined based on a plurality of movements of a subject. For example, the plurality of movements may comprise one or more of a set of walking movements, a set of balancing movements, a set of reflex movements, or a set of motor speed movements. In an example, the one or more neurological assessments may be determined based on one or more cognitive scores. For example, the one or more cognitive scores may comprise one or more of an action score, a rhythm score, or a function score. Machine learning and/or artificial intelligence may be used to predict the one or more neurological assessments of a subject and/or the one or more cognitive scores.

As shown in FIG. 1, the system 100 may comprise a computing device 102 (e.g., a user device, a server, a cloud device, a central computing device, etc.). The computing device 102 may be configured to receive motion data 104 associated with a plurality of movements of one or more subjects. As an example, the computing device 102 may receive the motion data 104 from one more sources such as one or more available databases. For example, the motion data 104 may comprise one or more of a walking data set, a balancing data set, a reflex data set, or a motor speed data set. For example, the motion data 104 may comprise one or more motion datasets, wherein the one or more datasets may comprise one or more adult subject datasets and/or one or more child subject datasets. The one or more datasets may comprise data associated with subjects that are healthy or clinically well characterized and may have had conventional cognitive assessments. The adult subject datasets may comprise data associated with subjects from 11 to 90 years old. The child datasets may comprise data associated with subjects from 5 to 10 years old.

The motion data 104 (e.g., one or more datasets) may be provided as inputs to a machine learning model 112 of the computing device 102. The machine learning model 112 may be trained based on the inputs in order or predict one or more neurological assessments 108 of a subject. In an example, one or more cognitive scores may be predicted in order to determine/predict the one or more neurological assessments 108.

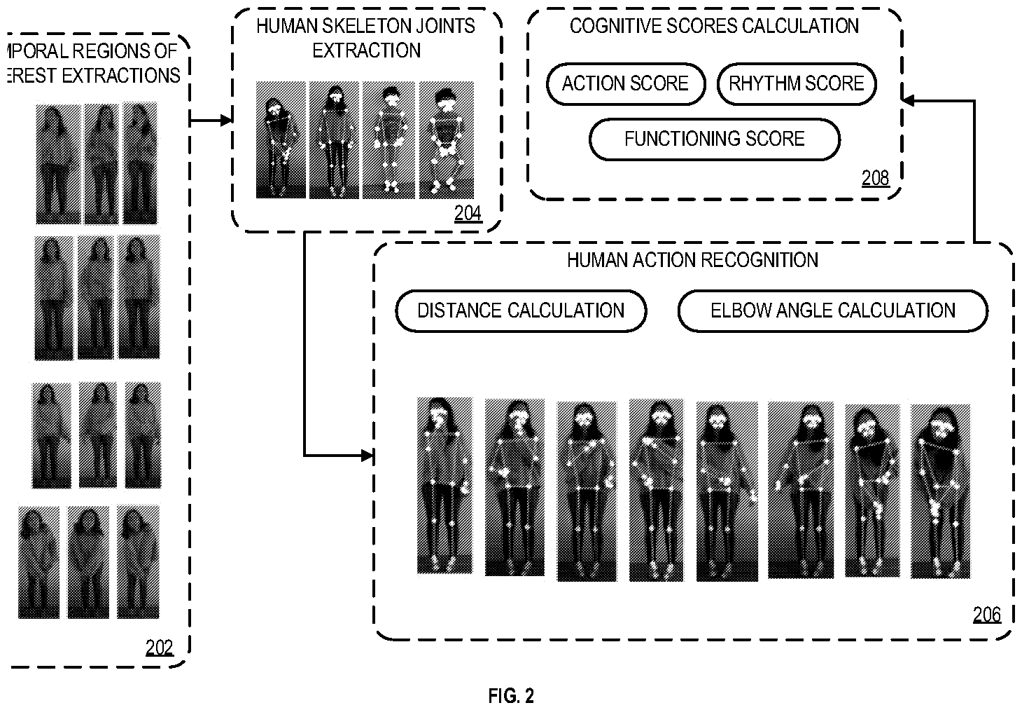

FIG. 2 shows an example model architecture 200 that may be used to determine one or more cognitive scores. The architecture 200 may comprise a temporal region of interest extraction module 202, a human skeleton joints extraction module 204, a human action recognition module 206, and a cognitive score calculation module 208. To efficiently segment videos that may be received of a subjects movements, temporal regions of interest may be extracted where the subject has a probability to practice an assigned task and touch a designated body part. Human skeleton joints may be extracted based on a Graph Convolutional Network (GCN) that may be configured to effectively capture spatial dynamics from a human skeleton. For example, the GCN may be configured to model human skeleton sequences as spatio-temporal graphs.

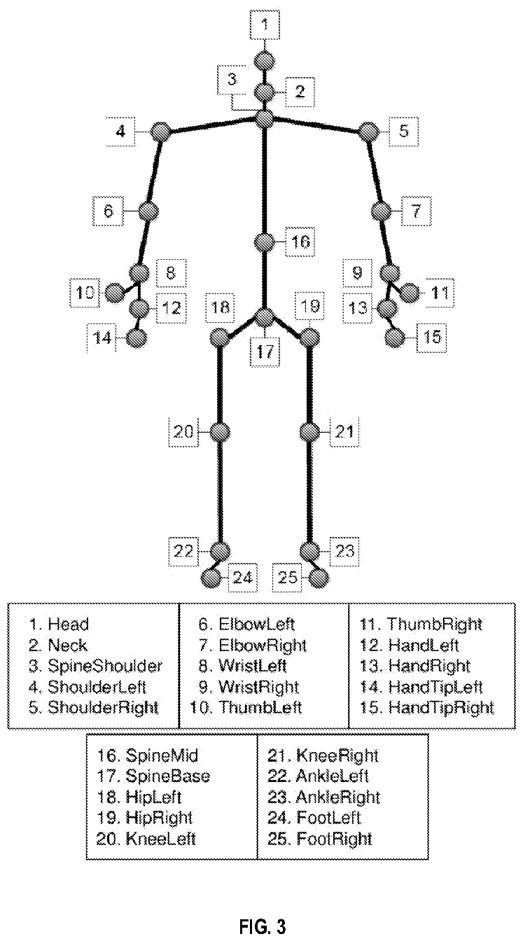

The human skeleton joints extraction module 204 may receive temporal regions of interest from the temporal region of interest extraction module 202 and extract the key points of the human body. For example, a plurality of key human body points may be extracted such as the key points shown in FIG. 3. As an example a plurality of tasks may be output to a subject, wherein cognition may be influenced via the plurality of tasks (e.g., physical activities). For example, a game comprising four trials with up to four paired behavioral rules may be output to the subject. The rules may comprise instructing a subject to touch his/her ears, shoulders, and/or knees. Initially a subject may respond naturally. Subsequently, the subject may be instructed to switch rules by responding in an opposite way (e.g., touch his/her knees when told to touch his/her ears). In an example, video content may be output to a subject instructing the subject to perform a task while following a song. Each trial may comprise varying trial segments, wherein in a single trial segment, the subject may be told to perform three touches based on the announced body part. A number of temporal regions of interest may be extracted from temporally segmented frames associated with a video to predict an action in each temporal region of interest. The temporal region of interest (TRI) may comprise a trial segment where the subject is instructed to touch a designated body part. In an example, a plurality of TRIs may be included in a video or game that may be output to a subject. In an example, the extraction of the TRIs may be performed via speech recognition to segment a video based on a song output to the subject.

As shown in FIG. 3, the human skeleton data may comprise a series of 3D coordinates of multiple skeleton joints, which may be estimated from 2D images captured of a subject. The human skeleton joints extraction module 204 may be configured to use a pose estimator model for determining multi-person and/or single-person pose estimations. The human skeleton joints extraction module 204 may extract data indicative of human joints to predict the location of all human skeleton key points, such as the human skeleton key points shown in FIG. 3.

The action recognition module 206 may determine an action for each temporal region of interest based on the extracted key points of the human body received from the human skeleton joints extraction module 204. One or more hand positions with respect to body part joints of interest may be monitored. Eight classes of “cross-your-body” tasks may be determined: class 1 may comprise the right hand against the left ear; class 2 may comprise the left hand against the right ear; class 3 may comprise the right hand against the left shoulder; class 4 may comprise the left hand against the right should; er class 5 may comprise the right hand against the left hip; class 6 may comprise the left hand against the right hip; class 7 may comprise the right hand against the left knee; and class 8 may comprise the left hand against the right knee. A determination of whether the subject's action complies with the expected motion may be evaluated. As an example, the actions may be determined based on a distance between hand joints and body part joints. As an example, the accuracy of determining the actions may be increased based on determining an elbow angle between the body part and an opposite hand.

The cognitive scores may be determined by the cognitive scores calculation module 208 based on the determined actions received from the action recognition module 206. For example, an action score, a rhythm score, and/or a function score may be determined based on the actions. The action score may be determined based on an amount of times that a subject touches a designated body part correctly at least once. The rhythm score may be determined based on an amount of times that a subject touches a desired body part within one second after receiving the instruction. The function score may be determined based on a total number of actions that a subject touches a designated body part. The cognitive scores may be translated to measure executive functioning which is a key factor in distinguishing self-regulation, response inhibition, working memory, co-ordination, and attention. For example, the cognitive scores may be evaluated to determine one or more neurological assessments of a subject.

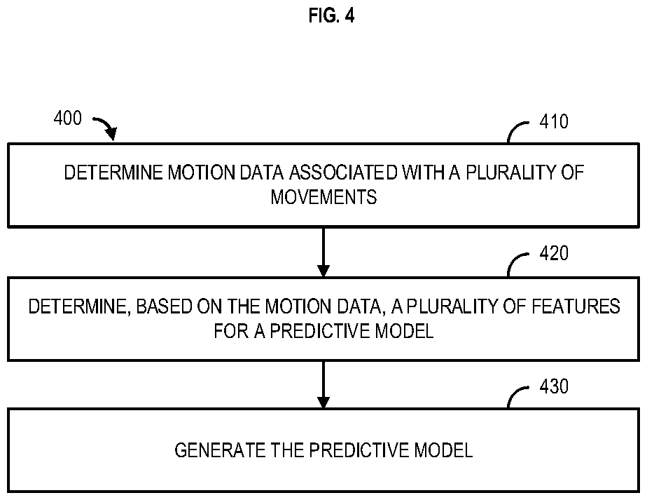

FIG. 4 shows a method 400 for generating a predictive model for determining/predicting one or more neurological assessments of a subject and/or one or more cognitive scores of a subject. Method 400 may comprise determining motion data associated with a plurality of movements at 410, determining, based on the motion data, a plurality of features for a predictive model at 420, and generating, based on the plurality of features, the predictive model at 430.

The motion data may comprise the plurality of movements. The plurality of movements in the motion data may be associated with one or more series of movements. Each series of movements of the plurality of movements may be labeled according to a predefined feature of a plurality of predefined features. The plurality of predefined features may include at least one neurological assessment of a motor function, balancing, reflex movement, sensory function, coordination, or gait.

Determining the motion data associated with the plurality of movements at 410 may comprise downloading/obtaining/receiving one or more movement data sets, obtained from various sources, including recent publications and/or publically available databases. The one or more movement data sets may comprise one or more of a walking data set, a balancing data set, a reflex data set, or a motor speed data set. In an example, the one or more movement data sets may comprise adult movement data sets and/or child movement data sets. The methods described herein may utilize the one or more movement data sets to improve identification of one or more neurological assessments and/or one or more cognitive scores.

Determining the motion data associated with the plurality of movements at 410 may further comprise determining baseline feature levels for each series of movements associated with the plurality of movements. The baseline feature levels for each series of movements may then be labeled as at least one predefined feature of the plurality of the predefined features. The plurality of predefined features may include at least one neurological assessment of a motor function, balancing, reflex movement, sensory function, coordination, or gait.

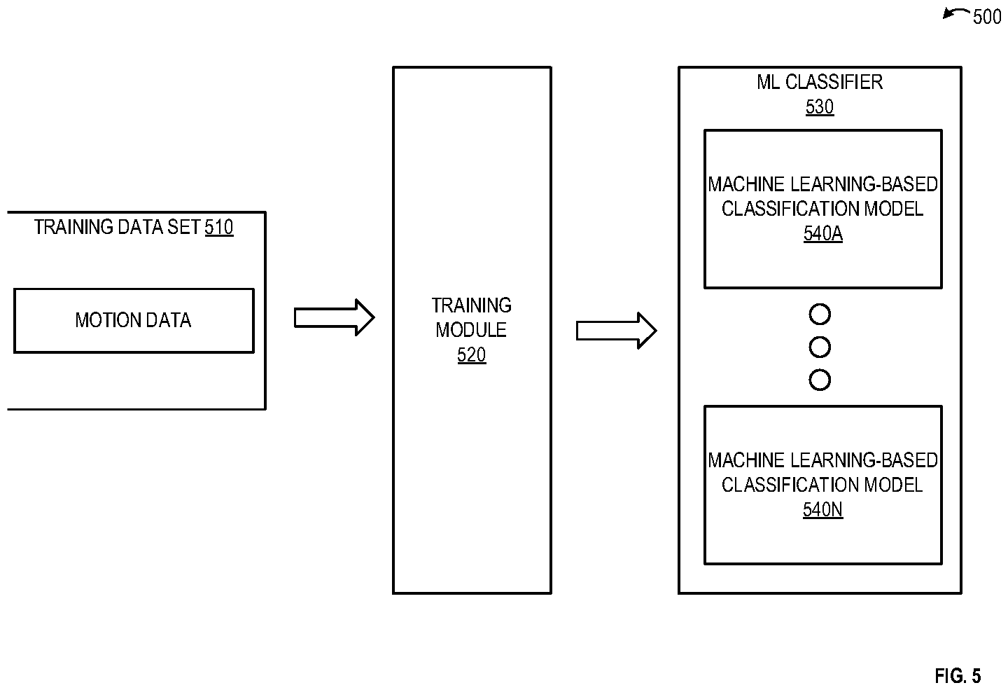

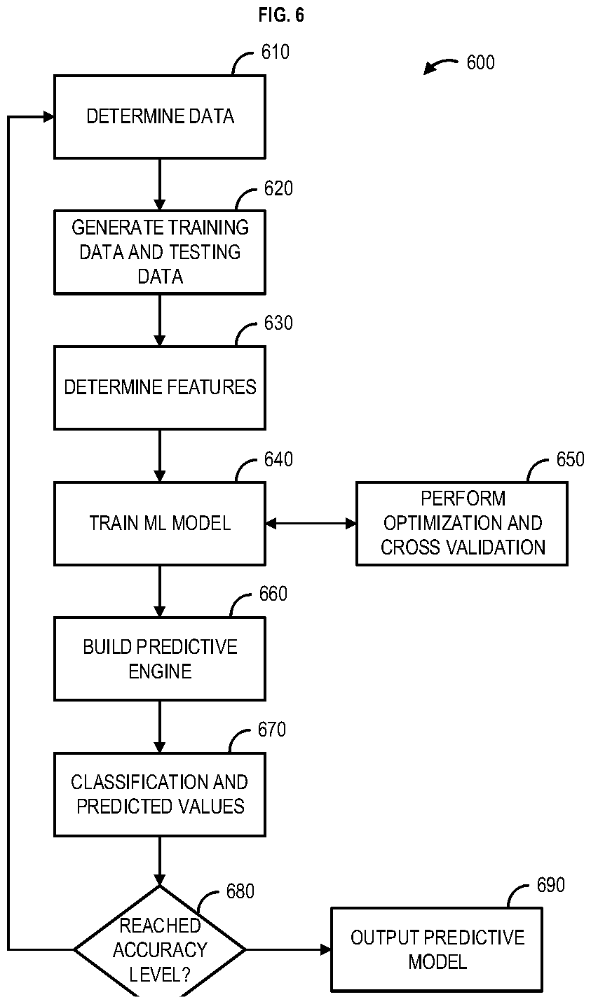

Determining, based on the motion data, a plurality of features for a predictive model at 420 and generating, based on the plurality of features, the predictive model at 430 are described with regard to FIG. 5 and FIG. 6.

A predictive model (e.g., a machine learning classifier) may be generated to classify one or more neurological assessments based on analyzing a plurality of observed movements of a subject. The one or more neurological assessments may comprise one or more of a deficiency of a subject's motor function, balancing, reflex movement, sensory function, coordination, or gait. In an example, the predictive model may be configured to determine one or more cognitive scores that may be used to classify the one or more neurological assessments. The one or more cognitive scores may comprise one or more of an action score, a rhythm score, or a function score. The predictive model may be trained according to the motion data (e.g., one or more movement data sets and/or baseline feature levels). The one or more movement data sets may contain data sets involving a subject's movements including a walking data set, a balancing data set, a reflex data set, or a motor data set. The baseline feature levels may relate to studies that involve observing a subject's movements according to predetermined tasks. In an example, one or more features of the predictive model may be extracted from one or more of the one or more movement data sets and/or the baseline feature levels.

FIG. 5 shows a system 500 that may be configured to use machine learning techniques to train, based on an analysis of one or more training data sets 510 by a training module 520, at least one machine learning-based classifier 530 that is configured to classify baseline feature data as being associated with one or more neurological assessments and/or one or more cognitive scores. In an example, the training data set 510 (e.g., the motion data) may comprise the one or more movement data sets and/or baseline feature levels. In an example, the training data set 510 may comprise labeled baseline feature levels. The labels may comprise predefined features associated with motor function, balancing, reflex movement, sensory function, coordination, or gait.

The training module 520 may train the machine learning-based classifier 530 by extracting a feature set from the motion data (e.g., one or more movement data sets and/or baseline feature levels) in the training data set 510 according to one or more feature selection techniques.

In an example, the training module 520 may extract a feature set from the training data set 510 in a variety of ways. The training module 520 may perform feature extraction multiple times, each time using a different feature-extraction technique. In an embodiment, the feature sets generated using the different techniques may each be used to generate different machine learning-based classification models 540. In an example, the feature set with the highest quality metrics may be selected for use in training. The training module 520 may use the feature set(s) to build one or more machine learning-based classification models 540A-540N that are configured to indicate whether or not new data is associated with a neurological assessment.

In an example, the training data set 510 may be analyzed to determine one or more series of movements that have at least one feature that may be used to predict a neurological assessment and/or a cognitive score. The one or more series of movements may be considered as features (or variables) in the machine learning context. The term “feature,” as used herein, may refer to any characteristic of a series, or group, of movements of a subject that may be used to determine whether the series of movements fall within one or more specific categories. By way of example, the features described herein may comprise one or more series of movements.

In an example, a feature selection technique may comprise one or more feature selection rules. The one or more feature selection rules may comprise a movement occurrence rule. The movement occurrence rule may comprise determining which movements in the training data set 510 occur over a threshold number of times and identifying those movements that satisfy the threshold as candidate features. For example, any movements that appear greater than or equal to 50 times in the training data set 510 may be considered as candidate features. Any movements appearing less than 50 times may be excluded from consideration as a feature.

In an example, the one or more feature selection rules may comprise a significance rule. The significance rule may comprise determining, from the baseline feature level data in the training data set 510, neurological assessment data. The neurological assessment data may include data associated with one or one more of motor function, balancing, reflex movement, sensory function, coordination, or gait. As the baseline feature level data in the training data set 510 are labeled according to a neurological assessment, the labels may be used to determine the neurological assessment data.

In an example, a single feature selection rule may be applied to select features or multiple feature selection rules may be applied to select features. For example, the feature selection rules may be applied in a cascading fashion, with the feature selection rules being applied in a specific order and applied to the results of the previous rule. For example, the movement occurrence rule may be applied to the training data set 510 to generate a first list of features. The significance rule may be applied to features in the first list of features to determine which features of the first list satisfy the significance rule in the training data set 510 and to generate a final list of candidate features.

The final list of candidate features may be analyzed according to additional feature selection techniques to determine one or more candidate feature signatures (e.g., series, or groups, of movements that may be used to predict a neurological assessment and/or a cognitive score). Any suitable computational technique may be used to identify the candidate feature signatures using any feature selection technique such as filter, wrapper, and/or embedded methods. In an example, one or more candidate feature signatures may be selected according to a filter method. Filter methods include, for example, Pearson's correlation, linear discriminant analysis, analysis of variance (ANOVA), chi-square, combinations thereof, and the like. The selection of features according to filter methods are independent of any machine learning algorithms. Instead, features may be selected on the basis of scores in various statistical tests for their correlation with the outcome variable (e.g., a neurological assessment and/or a cognitive score).

In an example, one or more candidate feature signatures may be selected according to a wrapper method. A wrapper method may be configured to use a subset of features and train a machine learning model using the subset of features. Based on the inferences that are drawn from a previous model, features may be added and/or deleted from the subset. Wrapper methods include, for example, forward feature selection, backward feature elimination, recursive feature elimination, combinations thereof, and the like. In an embodiment, forward feature selection may be used to identify one or more candidate feature signatures. Forward feature selection is an iterative method that begins with no feature in the machine learning model. In each iteration, the feature which best improves the model is added until an addition of a new variable does not improve the performance of the machine learning model. In an example, backward elimination may be used to identify one or more candidate feature signatures. Backward elimination is an iterative method that begins with all features in the machine learning model. In each iteration, the least significant feature is removed until no improvement is observed on removal of features. In an example, recursive feature elimination may be used to identify one or more candidate feature signatures. Recursive feature elimination is a greedy optimization algorithm which aims to find the best performing feature subset. Recursive feature elimination repeatedly creates models and keeps aside the best or the worst performing feature at each iteration. Recursive feature elimination constructs the next model with the features remaining until all the features are exhausted. Recursive feature elimination then ranks the features based on the order of their elimination.

In an example, one or more candidate feature signatures may be selected according to an embedded method. Embedded methods combine the qualities of filter and wrapper methods. Embedded methods include, for example, Least Absolute Shrinkage and Selection Operator (LASSO) and ridge regression which implement penalization functions to reduce overfitting. For example, LASSO regression performs L1 regularization which adds a penalty equivalent to absolute value of the magnitude of coefficients and ridge regression performs L2 regularization which adds a penalty equivalent to square of the magnitude of coefficients.

After the training module 520 has generated a feature set(s), the training module 520 may generate a machine learning-based classification model 540 based on the feature set(s). Machine learning-based classification model, may refer to a complex mathematical model for data classification that is generated using machine-learning techniques. In an example, this machine learning-based classifier may include a map of support vectors that represent boundary features. By way of example, boundary features may be selected from, and/or represent the highest-ranked features in, a feature set.

In an example, the training module 520 may use the feature sets extracted from the training data set 510 to build a machine learning-based classification model 540A-540N for each classification category (e.g., neurological assessment and/or cognitive score). In an example, the machine learning-based classification models 540A-540N may be combined into a single machine learning-based classification model 540. In an example, the machine learning-based classifier 530 may represent a single classifier containing a single or a plurality of machine learning-based classification models 540 and/or multiple classifiers containing a single or a plurality of machine learning-based classification models 540.

The extracted features (e.g., one or more candidate features and/or candidate features signatures derived from the final list of candidate features) may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like. The resulting machine learning-based classifier 530 may comprise a decision rule or a mapping that uses the expression levels of the features in the candidate feature signature to predict a neurological assessment.

The candidate feature signature and the machine learning-based classifier 530 may be used to predict the neurological assessment and/or cognitive score statuses in the testing data set. In an example, the result for each test includes a confidence level that corresponds to a likelihood or a probability that the corresponding test predicted a neurological assessment and/or a cognitive score status. The confidence level may be a value between zero and one that represents a likelihood that the corresponding test is associated with a neurological assessment and/or a cognitive score status. In an example, when there are two or more statuses (e.g., two or more neurological assessments and/or two or more cognitive scores), the confidence level may correspond to a value p, which refers to a likelihood that a particular test is associated with a first status. In this case, the value 1−p may refer to a likelihood that the particular test is associated with a second status. In general, multiple confidence levels may be provided for each test and for each candidate feature signature when there are more than two statuses. A top performing candidate feature signature may be determined by comparing the result obtained for each test with known neurological assessment and/or cognitive score statuses for each test. In general, the top performing candidate feature signature will have results that closely match the known neurological assessment and/or the known cognitive score statuses.

The top performing candidate feature signature may be used to predict the neurological assessment and/or the cognitive score status of a subject. For example, baseline feature data for a potential patient may be determined/received. The baseline feature data for the potential subject may be provided to the machine learning-based classifier 530 which may, based on the top performing candidate feature signature, predict/determine a neurological assessment and/or a cognitive score of the subject. Depending on the predicted/determined neurological assessment and/or cognitive score, the subject may be treated accordingly.

FIG. 6 shows a flowchart of an example training method 600 for generating the machine learning-based classifier 530 using the training module 520. The training module 520 can implement supervised, unsupervised, and/or semi-supervised (e.g., reinforcement based) machine learning-based classification models 540. As an example, the method 600 shown in FIG. 6 may comprise a supervised learning method. Although variations of training method 600 are discussed below, other training methods can be analogously implemented to train unsupervised and/or semi-supervised machine learning models.

The training method 600 may determine (e.g., access, receive, retrieve, etc.) motion data (e.g., one or more movement data sets and/or baseline feature levels) of one or more subjects at 610. The motion data may contain one or more datasets, wherein each dataset may be associated with a particular study. Each study may involve different subject populations (e.g., adult and/or child populations), although it is contemplated that some subject overlap may occur. In an example, each dataset may include a labeled list of predetermined features. In an example, each dataset may comprise labeled feature data. The labels may be associated with one or more neurological assessments associated with motor function, balancing, reflex movement, sensory function, coordination, or gait.

The training method 600 may generate, at 620, a training data set and a testing data set. The training data set and the testing data set may be generated by randomly assigning labeled feature data of individual features from the motion data to either the training data set or the testing data set. In an example, the assignment of the labeled feature data of individual features may not be completely random. In an example, only the labeled feature data for a specific study may be used to generate the training data set and the testing data set. In an example, a majority of the labeled feature data for the specific study may be used to generate the training data set. For example, 75% of the labeled feature data for the specific study may be used to generate the training data set and 25% may be used to generate the testing data set. In an example, only the labeled feature data for the specific study may be used to generate the training data set and the testing data set.

The training method 600 may determine (e.g., extract, select, etc.), at 630, one or more features that can be used by, for example, a classifier to differentiate among different classifications (e.g., different neurological assessments). The one or more features may comprise a series of movements. In an example, the training method 600 may determine a set of features from the motion data. In an example, a set of features may be determined from motion data from a study different than the study associated with the labeled feature data of the training data set and the testing data set. In other words, motion data from the different study (e.g., curated movement data sets) may be used for feature determination, rather than for training a machine learning model. In an example, the training data set may be used in conjunction with the motion data from the different study to determine the one or more features. The motion data from the different study may be used to determine an initial set of features, which may be further reduced using the training data set.

The training method 600 may train one or more machine learning models using the one or more features at 640. In an example, the machine learning models may be trained using supervised learning. In an example, other machine learning techniques may be employed, including unsupervised learning and semi-supervised. The machine learning models trained at 640 may be selected based on different criteria depending on the problem to be solved and/or data available in the training data set. For example, machine learning classifiers can suffer from different degrees of bias. Accordingly, more than one machine learning model may be trained at 640, optimized, improved, and cross-validated at 650.

The training method 600 may select one or more machine learning models to build a predictive model at 660 (e.g., a machine learning classifier). The predictive model may be evaluated using the testing data set. The predictive model may analyze the testing data set and generate classification values and/or predicted values at 670. Classification and/or prediction values may be evaluated at 680 to determine whether such values have achieved a desired accuracy level. Performance of the predictive model may be evaluated in a number of ways based on a number of true positive, false positive, true negative, and/or false negative classifications of the plurality of data points indicated by the predictive model. For example, the false positives of the predictive model may refer to a number of times the predictive model incorrectly classified a subject's movements. Conversely, the false negatives of the predictive model may refer to a number of times the machine learning model determined that a neurological assessment and/or a cognitive score was not associated with one or more of a subject's movements when, in fact, the subject's movements were associated with a neurological assessment and/or a cognitive score. True negatives and true positives may refer to a number of times the predictive model correctly classified one or more neurological assessments and/or one or more cognitive scores. Related to these measurements are the concepts of recall and precision. Generally, recall refers to a ratio of true positives to a sum of true positives and false negatives, which quantifies a sensitivity of the predictive model. Similarly, precision refers to a ratio of true positives a sum of true and false positives.

When such a desired accuracy level is reached, the training phase ends and the predictive model may be output at 690; when the desired accuracy level is not reached, however, then a subsequent iteration of the training method 600 may be performed starting at 610 with variations such as, for example, considering a larger collection of movement data.

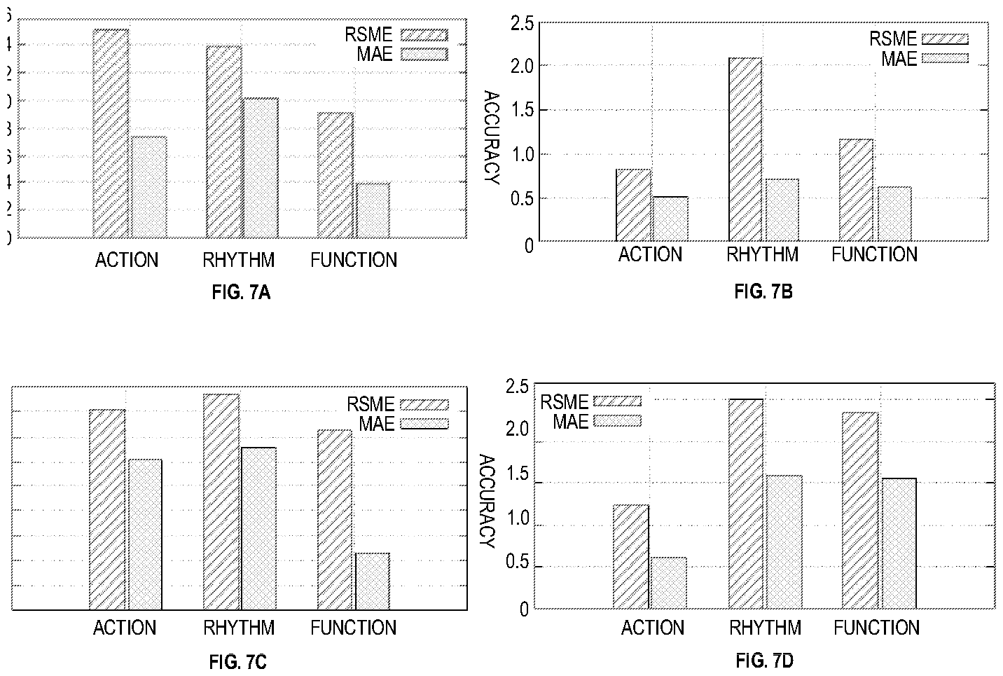

FIGS. 7A-7D show example prediction results. Subjects may be analyzed based on instructions to perform one or more cognitive tasks. In an example, for a first trial, subjects may be instructed to touch the same body part, for a second trial, or set of trials, subjects may be instructed to perform progressively cognitively demanding tasks, and for a third trial subjects may be required to touch their knees when the subjects are instructed to touch their ears and vice versa. In an example, for a third trial, subjects may be instructed to switch shoulders and hips while a fourth trial may include the rules of the second and third trials. The performance of the predictive model may be evaluated to determine the accuracy of predicting one or more neurological assessments. In an example, the predictive model may be evaluated to determine the accuracy of the one or more predicted cognitive scores. FIGS. 7A-7D show prediction results for children based on example trials such as trials 1-4 discussed above. A Root Mean Square Error (RMSE) and a Mean Absolute Error (MAE) may be measured for each predicted cognitive score. FIGS. 7A-7D show that the function score prediction is highly related to the action score prediction. For example, although the actions performed by children may be imprecise, the function and action scores may be impacted.

TABLE I

| | Action Score | | Rhythm Score | | Function Score | |
|---|---|---|---|---|---|---|
| Method | RMSE | MAE | RMSE | MAE | RMSE | MAE |
| DTGRM | 1.652 | 0.791 | 1.521 | 1.192 | 0.101 | 0.498 |
| MSTCN | 1.532 | 0.738 | 1.422 | 1.132 | 0.925 | 0.412 |
| ASRF | 1.743 | 0.818 | 1.635 | 1.254 | 1.132 | 0.52 |
| MSTCN | 2.051 | 1.215 | 1.982 | 1.458 | 1.532 | 0.891 |
| MIA | 1.312 | 0.635 | 1.798 | 0.912 | 0.707 | 0.250 |

In an example, several learning-based methods may be used to evaluate the prediction model. For example, a dilated temporal graph reasoning module (DTGRM) may be configured to use GCNs to model temporal relations of videos capturing a subject's movements, a multi-stage temporal convolutional network (MSTCN) may be configured to use an auxiliary self-supervised task to find correct and in-correct temporal relations of videos capturing a subject's movements, an action refinement framework (ASRF) model may be used, wherein the ASRF may be used to alleviate over-segmentation errors by detecting action boundaries, and an MSTCN++ may be used as an improvement over MSTCN to generate frame level predictions using a dual dilated layer that combines small and large receptive fields. Table I shows the RMSE and MAE for each predictive cognitive score determined by each model. Table I further shows that the function score prediction is highly related to the action score prediction. Although the actions may be mis-identified, the function and action scores may be impacted.

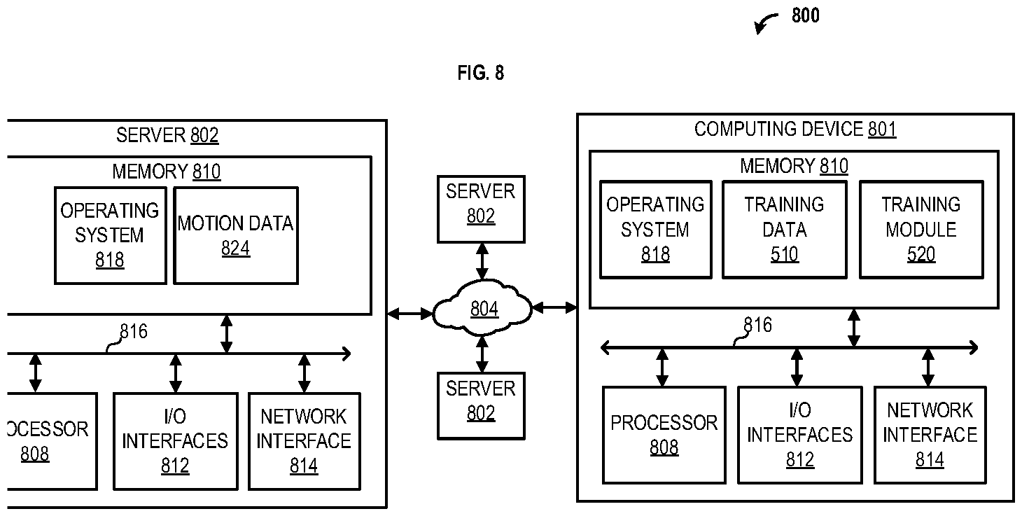

FIG. 8 shows a block diagram of an example system 800, for determining one or more neurological assessments and/or one or more cognitive scores. The system 800 may comprise a computing device 801 and a server 802 connected via a network 804. As an example, some or all steps of any described method may be performed on a computing device as described herein. The computing device 801 may comprise one or multiple computers configured to store one or more of the training module 520, training data 510 (e.g., labeled baseline feature data and/or curated movement set data), and the like. The server 802 may comprise one or multiple computers configured to store motion data 824 (e.g., curated movement set data). Multiple servers 802 may communicate with the computing device 801 via the network 804.

The computing device 801 and the server 802 may comprise a digital computer that may comprise a processor 808, memory system 810, input/output (I/O) interfaces 812, and network interfaces 814. In an example, the computing device 801 may comprise one or more of a depth camera device, an image capturing device, a smart television, a tablet computer, a desktop, or any device configured to receive and process motion data associated with a subject. The processor 808, the memory system 810, input/output (I/O) interfaces 812, and network interfaces 814 may be communicatively coupled via a local interface 816. The local interface 816 may comprise one or more buses or other wired or wireless connections, as is known in the art. The local interface 816 may comprise additional elements, which are omitted for simplicity, such as controllers, buffers (caches), drivers, repeaters, and receivers, to enable communications. Further, the local interface may include address, control, and/or data connections to enable appropriate communications among the processor 808, the memory system 810, the input/output (I/O) interfaces 812, and the network interfaces 814.

The processor 808 may comprise a hardware device for executing software that may be stored in memory system 810. The processor 808 may comprise any custom made or commercially available processor, a central processing unit (CPU), an auxiliary processor among several processors associated with the computing device 801 and the server 802, a semiconductor-based microprocessor (in the form of a microchip or chip set), or generally any device for executing software instructions. When the computing device 801 and/or the server 802 is in operation, the processor 808 may be configured to execute software stored within the memory system 810, to communicate data to and from the memory system 810, and to generally control operations of the computing device 801 and the server 802 pursuant to the software.

The I/O interfaces 812 may comprise one or more interfaces for receiving user input from, and/or for providing system output to, one or more devices or components. User input can be provided via, for example, a keyboard and/or a mouse. System output may be provided via a display device and a printer (not shown). I/O interfaces 412 may include, for example, a serial port, a parallel port, a Small Computer System Interface (SCSI), an infrared (IR) interface, a radio frequency (RF) interface, and/or a universal serial bus (USB) interface.

The network interface 814 may be configured to transmit and receive from the computing device 801 and/or the server 802 via the network 804. The network interface 814 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 814 may include address, control, and/or data connections to enable appropriate communications on the network 804.

The memory system 810 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 810 may incorporate electronic, magnetic, optical, and/or other types of storage media. In an example, the memory system 810 can have a distributed architecture, where various components are situated remote from one another, but can be accessed by the processor 808.

The software in memory system 810 may include one or more software programs, each of which comprises an ordered listing of executable instructions for implementing logical functions. The software in the memory system 810 of the computing device 801 may comprise the training module 520 (or subcomponents thereof), the training data 510, and a suitable operating system (O/S) 818. The software in the memory system 810 of the server 802 may comprise the training data 510 and a suitable operating system (O/S) 818. The operating system 818 may be configured to control the execution of other computer programs and provides scheduling, input-output control, file and data management, memory management, and communication control and related services.

FIG. 8 shows application programs and other executable program components such as the operating system 818 as discrete blocks, although it is recognized that such programs and components can reside at various times in different storage components of the computing device 801 and/or the server 802. As an example, the training module 520 may be stored on or transmitted across some form of computer readable media. Any of the disclosed methods can be performed by computer readable instructions embodied on computer readable media. Computer readable media can be any available media that can be accessed by a computer. For example, computer readable media may comprise “computer storage media” and “communications media.” “Computer storage media” may comprise volatile and non-volatile, removable and non-removable media implemented in any method or technology for storage of information such as computer readable instructions, data structures, program modules, or other data. In an example, computer storage media may comprise RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

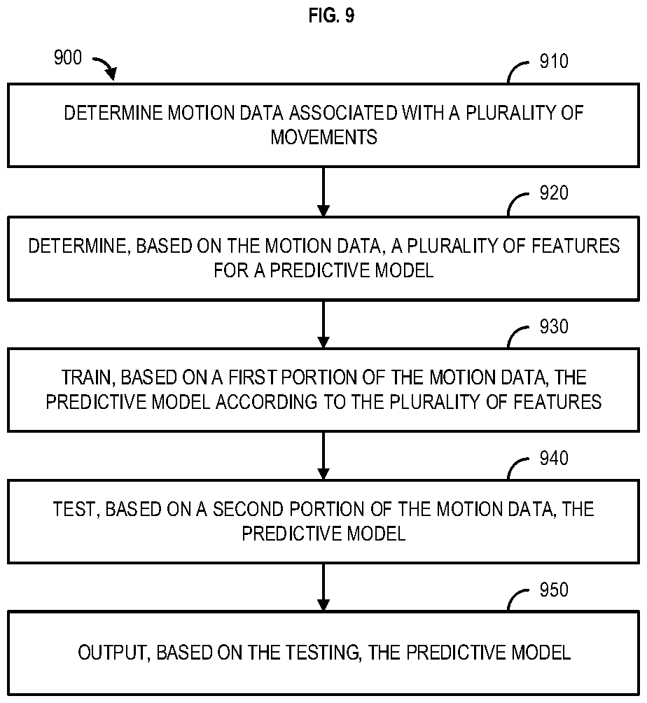

FIG. 9 shows a flowchart of an example method 900. Method 900 may be implemented in whole, or in part, by computing device 801, server 802, or any other suitable device. For example, method 900 may be implemented in whole, or in part, by the training module 520. At step 910, motion data associated with a plurality of movements may be determined. The motion data may comprise movement data from a plurality of different data sets. The motion data may be retrieved from a public or private data source and the plurality of movements may comprise one or more of a walking data set, a balancing data set, a reflex data set, or a motor speed data set.

As an example, determining the motion data associated with the plurality of movements may further comprise determining baseline feature levels for each series of movements associated with the plurality of movements. The baseline feature levels for each series of movements may then be labeled as at least one predefined feature of the plurality of the predefined features. The plurality of predefined features may include at least one neurological assessment of motor function, balancing, reflex movement, sensory function, coordination, or gait.

At step 920, a plurality of features for a predictive model may be determined based on the motion data. As an example, determining, based on the motion data, the plurality of features for the predictive model may comprise determining, from the motion data, features present in two or more of the plurality of different movement data sets as a first set of candidate movements, determining, from the motion data, features of the first set of candidate movements that satisfy a first threshold value as a second set of candidate movements, and determining, from the motion data, features of the second set of candidate movements that satisfy a second threshold value as a third set of candidate movements, wherein the plurality of features comprises the third set of candidate movements. In an embodiment, determining, based on the motion data, the plurality of features for the predictive model may comprise determining, for the third set of candidate movements, a feature score for each of the plurality of movements associated with the third set of candidate movements, and determining, based on the feature score, a fourth set of candidate movements, wherein the plurality of features comprises the fourth set of candidate movements.

At step 930, the predictive model may be trained according to the plurality of features based on a first portion of the motion data. For example, training, based on a first portion of the motion data, the predictive model according to the plurality of features may result in determining a feature signature indicative of a neurological assessment and/or a cognitive score.

At step 940, the predictive model may be tested based on a second portion of the motion data. At step 950, the predictive model may be output based on the testing. The predictive model may be configured to output a prediction indicative of one or more neurological assessments. The one or more neurological assessments may comprise one or more of motor function, balancing, reflex movement, sensory function, coordination, or gait. In an example, the predictive model may be configured to output one or more cognitive scores that may be used to determine the one or more neurological assessments. The one or more cognitive scores may comprise one or more of an action score, a rhythm score, or a function score.

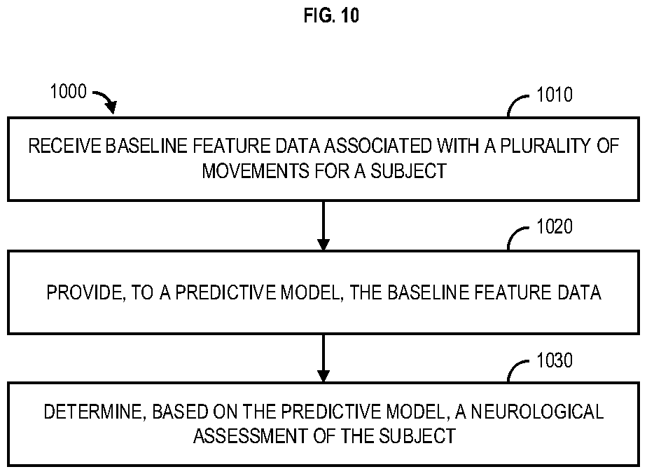

FIG. 10 shows a flowchart of an example method 1000. Method 1000 may be implemented in whole, or in part, by computing device 801, server 802, or any other suitable device. For example, method 1000 may be implemented in whole, or in part, by the training module 520. At step 1010, baseline feature data associated with a plurality of movements for a subject may be received. The plurality of movements may be determined from a plurality of observed movements of a subject. At step 620, the baseline feature data may be provided to a predictive model. At step 630, one or more neurological assessments of the subject may be determined based on the predictive model. The one or more neurological assessments may comprise one or more of motor function, balancing, reflex movement, sensory function, coordination, or gait. As an example, a subject may be treated with a therapeutic treatment based on the predicted neurological assessment. In an example, one or more cognitive scores may be determined based on the predictive model. The one or more cognitive scores may comprise one or more of an action score, a rhythm score, or a function score.

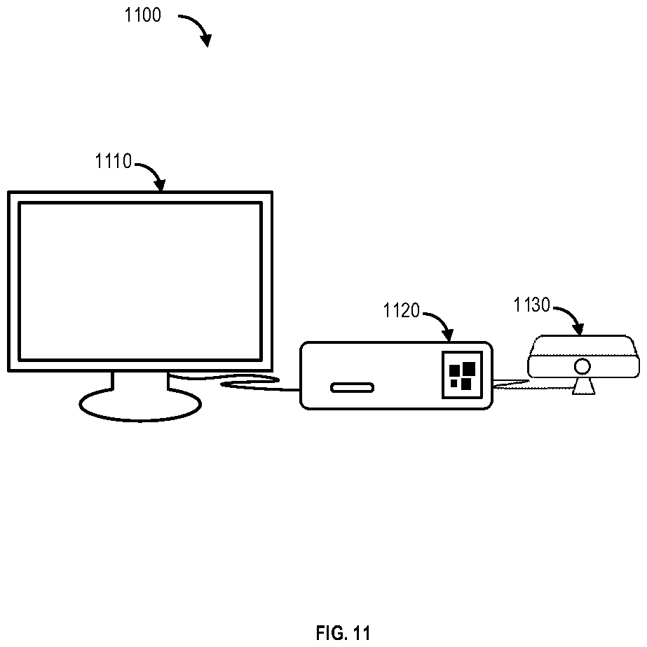

As an example, receiving baseline feature data associated with a plurality of movements for a subject may comprise detecting a subject's movements while performing an embodied cognitive task presented to the subject via a computing system, such as computing system 1100 shown in FIG. 11.

FIG. 11 shows an example computing system 1100 that may be configured to present an embodied cognitive task to a subject. The computing system 1100 may comprise a display device 1110, a computing subsystem/device 1120, a motion capture device 1130, and any other suitable devices. Computing subsystem/device 1120 may comprise a video game console, mainframe computer, server computer, desktop computer, laptop computer, tablet computer, home-entertainment computer, network computing device, mobile computing device, mobile communication device (e.g., smart phone), etc.

The display device 1110 may be configured to output an embodied cognitive task based on instructions from the computing subsystem/device 1120. The embodied cognitive task may comprise a video game, a graphical user interface (GUI), and the like. The display device 1110 may comprise one or more display devices utilizing virtually any type of technology. Such display devices may be combined with the computing subsystem/device 1120 in a shared enclosure, or such display devices may be peripheral display devices such as the display device 1110 shown in FIG. 11.

The embodied cognitive task may be output to a subject in the form of a video game via the display device 1110 of the computing system 1100. The video game may include engaging elements (e.g. music, art work, adaptivity, feedback, rewards/incentives, and/or the like). The embodied cognitive task may be designed such that the cognitive component of the task targets (e.g., interrogates) one or more cognitive abilities of the subject. For example, the cognitive component may target a cognitive ability of the subject selected from working memory, attention, task-switching, goal management, target search, target discrimination, response inhibition, and any combination thereof. The Automated Test of Embodied Cognition (“ATEC”) may include 18 different tasks (with 38 distinct trials) from balance and rapid sequencing tasks to complex tasks involving executive functions. The tasks may be presented in a hierarchical sequence of increasing difficulty. This hierarchy of item difficulty is common in neurocognitive assessments and makes it more likely that the test will be sensitive at low and at higher levels of ability (reducing basement and ceiling effects). Because the embodied cognitive task is a cognitive task that requires a physical response from the subject, in certain aspects the subject's performance on the cognitive component is assessed based on the detected body movement of the subject. The body movement may indicate the subject's reaction time to the task, accuracy on the task, or both.

The motion capture device 1130 may comprise a video camera, such as a web camera, an RGB video camera, and/or a depth camera (e.g., Kinect camera). Movements made by the subject may be captured in a sequence of images by the motion capture device 1130 and then processed by the computing subsystem/device 1120. The motion capture device 1130 may be configured to detect and perform gesture recognition associated with a subject's movements and monitor direction and relative distance of movements by a subject. In an example, the motion capture device 1130 may be configured to detect a subject's pupillary response while responding to the embodied cognitive task.

In an example, the motion capture device 1130 may be configured to capture images of the subject's movements. The captured images may be used to locate feature points of interest on the subject's body. For example, feature points of interest may include joints and locations corresponding to the user's left foot, left knee, left hip, left hand, left elbow, left shoulder, head, right shoulder, right elbow, right hand, right hip, right knee, right foot, and/or any other useful feature points for detecting movement of the subject.

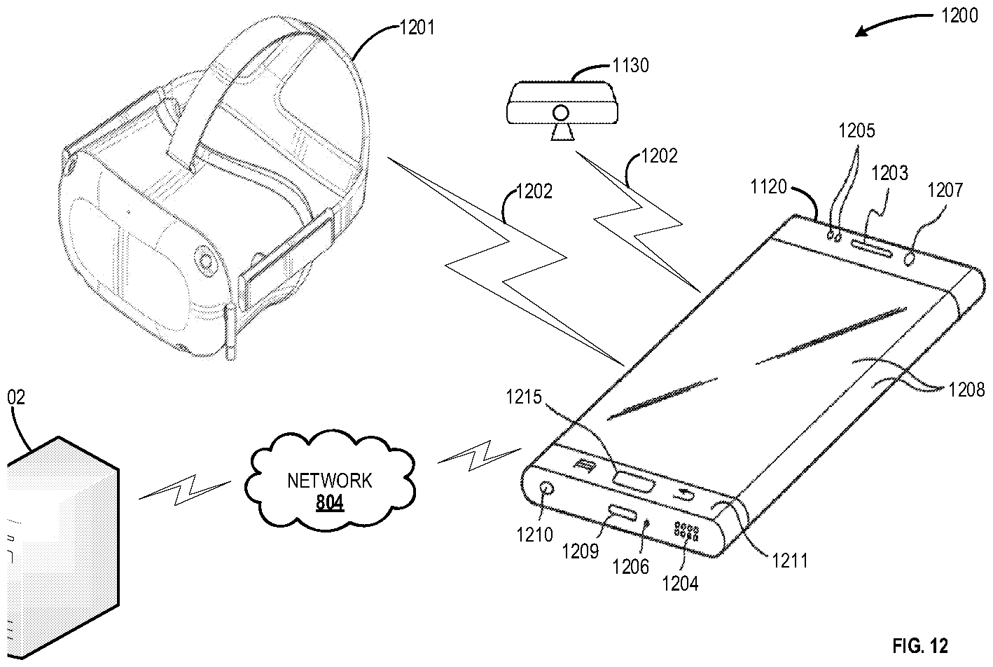

In an example, the computing system 1100 may comprise a virtual reality or augmented reality headset such as in computing system 1200 shown in FIG. 12. The computing system 1200 may comprise server 802, computing subsystem/device 1120, motion capture device 1130, and a virtual reality or augmented reality headset 1201. The computing subsystem/device 1120 and the headset 1201 may be communicatively coupled via a near field communication technology 1202 such as Bluetooth or WiFi. The headset 1201 may be configured to send data to the computing subsystem/device 1120. The computing subsystem/device 1120 may determine, via various sensors, image data, orientation data, and the like. The computing subsystem/device 1120 may further transmit said data to the server 802.

The computing subsystem/device 1120 and the headset 1201 may be communicatively coupled to the server 802 via network 804. The computing subsystem/device 1120 may comprise a display 1208, a housing (or a body) 1211 to which the display 1208 is coupled while the display 1208 is seated therein, and an additional device formed on the housing 1211 to perform the function of the computing subsystem/device 1120. As an example, the additional device may comprise a first speaker 1203, a second speaker 1204, a microphone 1206, sensors (for example, a front camera module 1207, an illumination sensor 1205, a rear camera module, or the like), communication interfaces (for example, a charging or data input/output port 1209 and an audio input/output port 1210), and a button 1215. In an example, when the computing subsystem/device 1120 and the headset 1201 are connected via a wired communication scheme, the computing subsystem/device 1120 and the headset 1201 may be connected based on at least some ports (for example, the data input/output port 1209) of the communication interfaces.

The display 1208 may comprise a flat display or a bended display (or a curved display) which may be folded or bent through a paper-thin or flexible substrate without damage. The bended display may be coupled to a housing 1211 to remain in a bent form. As an example, the computing subsystem/device 1120 may be implemented as a display device, which can be freely folded and unfolded such as a flexible display, including the bended display. As an example, in a Liquid Crystal Display (LCD), a Light Emitting Diode (LED) display, an Organic LED (OLED) display, or an Active Matrix OLED (AMOLED) display, the display 1208 may replace a glass substrate surrounding liquid crystal with a plastic film to assign flexibility to be folded and unfolded.

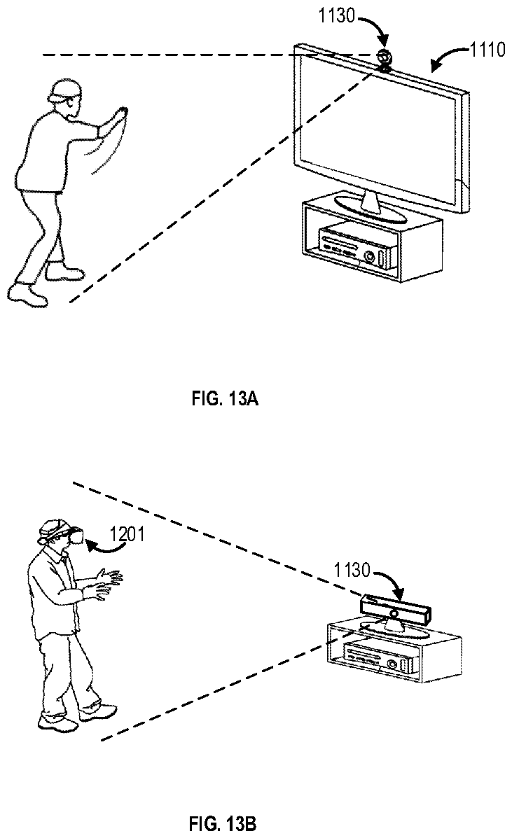

FIGS. 13A-13B show example scenarios of a subject performing the embodied cognitive tasks via computing systems 1100 and 1200. FIG. 13A shows an example scenario wherein a subject may perform the designated embodied cognitive tasks wherein the instructions may be output via display device 1130. FIG. 13B shows an example scenario wherein a subject may perform the designated embodied cognitive tasks wherein the instruction may be output via headset 1201 (e.g., via virtual reality or augmented reality). As the subject performs the designated embodied cognitive tasks the motion capture device 1130 may capture the subject's movements. The Automated Test of Embodied Cognition (“ATEC”) may include 18 different tasks (with 38 distinct trials) from balance and rapid sequencing tasks to complex tasks involving executive functions. The tasks may be presented in a hierarchical sequence of increasing difficulty. Because the embodied cognitive task is a cognitive task that requires a physical response from the subject, in certain aspects the subject's performance on the cognitive component is assessed based on the detected body movement of the subject. The body movement may indicate the subject's reaction time to the task, accuracy on the task, or both. The motion capture device 1130 may be configured to capture images of the subject's movements. The captured images may be used to locate feature points of interest on the subject's body. For example, the feature points of interest may include joints and locations corresponding to the user's left foot, left knee, left hip, left hand, left elbow, left shoulder, head, right shoulder, right elbow, right hand, right hip, right knee, right foot, and/or any other useful feature points for detecting movement of the subject. The motion capture device 1130 may send the captured images of the subject's movements to the computing subsystem/device 1120 for further processing.

For example, classification and/or evaluation of the subject's recorded movements may be accomplished by providing the captured images of the subject's movements to the predictive model (e.g., stored on the computing subsystem/device 1120). The predictive model may be used to determine one or more neurological assessments of the subject based on the captured images of the subject's movements. For example, the predictive model may be used to determine one or more cognitive scores (e.g., action score, rhythm score, and/or function score) associated with the subject's movements based on the captured images of the subject's movements.

The method 1000 may further comprise training the predictive model.

Training the predictive model may comprise determining motion data associated with the plurality of movements, wherein the plurality of movements include one or more series of movements, wherein each series of movements of the plurality of movements is labeled according to a predefined feature of a plurality of predefined features, determining, based on the motion data, a plurality of features for the predictive model, training, based on a first portion of the motion data, the predictive model according to the plurality of features, testing, based on a second portion of the motion data, the predictive model, and outputting, based on the testing, the predictive model.

The motion data may comprise one or more movement data sets retrieved from a public data source and the one or more movement data sets may comprise one or more of a walking data set, a balancing data set, a reflex data set, or a motor speed data set. The motion data may be comprised of movement data from a plurality of different data sets.

Determining, based on the motion data, the plurality of features for the predictive model may comprise determining, from the motion data, features present in two or more of the plurality of different movement data sets as a first set of candidate movements, determining, from the motion data, features of the first set of candidate movements that satisfy a first threshold value as a second set of candidate movements, and determining, from the motion data, features of the second set of candidate movements that satisfy a second threshold value as a third set of candidate movements, wherein the plurality of features comprises the third set of candidate movements.

Determining, based on the motion data, the plurality of features for the predictive model may comprise determining, for the third set of candidate movements, a feature score for of the plurality of movements associated with the third set of candidate movements, and determining, based on the feature score, a fourth set of candidate movements, wherein the plurality of features comprises the fourth set of candidate movements.

Determining the motion data associated with the plurality of movements may comprise determining baseline feature levels for each series of movements associated with the plurality of movements, labeling the baseline feature levels for each series of movements associated with the plurality of movements as at least one predefined feature of the plurality of predefined features, and generating, based on the labeled baseline feature levels, the motion data.

Training, based on the first portion of the motion data, the predictive model according to the plurality of features results in determining a feature signature indicative of a neurological assessment.

Embodiment 1: A method comprising: determining motion data associated with a plurality of movements, wherein the plurality of movements include one or more series of movements, wherein each series of movements of the plurality of movements is labeled according to a predefined feature of a plurality of predefined features, determining, based on the motion data, a plurality of features for a predictive model, training, based on a first, testing, based on a second portion of the motion data, the predictive model, and outputting, based on the testing, the predictive model.

Embodiment 2: The embodiment as in any one of the preceding embodiments wherein determining the motion data associated with a plurality of movements comprises retrieving the motion data from a public data source.

Embodiment 3: The embodiment as in any one of the preceding embodiments, wherein the plurality of movements comprise one or more of a walking set, a balancing set, a reflex set, or a motor speed set.

Embodiment 4: The embodiment as in any one of the preceding embodiments, wherein determining the motion data associated with the plurality of movements comprises: determining, based on the plurality of movements, one or more movement data sets that comprise at least one movement of the plurality of movements, and generating, based on the one or more movement data sets, the motion data.

Embodiment 5: The embodiment as in any one of the preceding embodiments wherein the motion data is comprised of movement data from a plurality of different movement data sets.

Embodiment 6: The embodiment as in any one of the preceding embodiments wherein determining the motion data associated with the plurality of movements comprises: determining baseline feature levels for each series of movements associated with the plurality of movements, labeling the baseline feature levels for each series of movements associated with the plurality of movements as at least one predefined feature of the plurality of the predefined features, and generating, based on the labeled baseline feature levels, the motion data.

Embodiment 7: The embodiment as in any one of the embodiments 5-6 wherein determining, based on the motion data, the plurality of features for the predictive model comprises: determining, from the motion data, features present in two or more of the plurality of different movement data sets as a first set of candidate movements, determining, from the motion data, features of the first set of candidate movements that satisfy a first threshold value as a second set of candidate movements, determining, from the motion data, features of the second set of candidate movements that satisfy a second threshold value as a third set of candidate movements, wherein the plurality of features comprises the third set of candidate movements.

Embodiment 8: The embodiment as in any one of the embodiments 5-7 wherein determining, based on the motion data, the plurality of features for the predictive model comprises: determining, for the third set of candidate movements, a feature score for each of the plurality of movements associated with the third set of candidate movements, and determining, based on the feature score, a fourth set of candidate movements, wherein the plurality of features comprises the fourth set of candidate movements.

Embodiment 9: The embodiment as in any one of the preceding embodiments wherein training, based on the first portion of the motion data, the predictive model according to the plurality of features results in determining a feature signature indicative of at least one of predefined feature of the plurality of predefined features.

Embodiment 10: The embodiment as in any one of the preceding embodiments wherein the plurality of features include at least one neurological assessment of one or more of motor function, balancing, reflex movement, sensory function, coordination, or gait.

Embodiment 11: A method comprising: receiving baseline feature data associated with a plurality of movements of a subject, wherein the plurality of movements are determined from a plurality of observed movements, providing, to a predictive model, the baseline feature data, and determining, based on the predictive model, a neurological assessment of the subject.

Embodiment 12: The embodiment as in the embodiment 11, wherein the neurological assessment comprises at least one of motor function, balancing, reflex movement, sensory function, coordination, or gait.

Embodiment 13: The embodiment as in any one of the embodiments 11-12 further comprising training the predictive model.

Embodiment 14: The embodiment as in any one of the embodiments 11-13, wherein training the predictive model comprises: determining motion data associated with the plurality of movements, wherein the plurality of movements include one or more series of movements, wherein each series of movements of the plurality of movements is labeled according to a predefined feature of a plurality of predefined features, determining, based on the motion data, a plurality of features for the predictive model, training, based on a first portion of the motion data, the predictive model according to the plurality of features, testing, based on a second portion of the motion data, the predictive model, and outputting, based on the testing, the predictive model.

Embodiment 15: The embodiment as in the embodiment 14 wherein determining the motion data associated with the plurality of movements comprises: determining, based on the plurality of movements, one or more movement data sets that comprise at least one movement of the plurality of movements, and generating, based on the one or more movement data sets, the motion data.

Embodiment 16: The embodiment as in the embodiments 14-15 wherein the motion data is comprised of movement data from a plurality of different movement data sets.

Embodiment 17: The embodiment as in the embodiments 14-16 wherein determining the motion data associated with the plurality of movements comprises: determining baseline feature levels for each series of movements associated with the plurality of movements, labeling the baseline feature levels for each series of movements associated with the plurality of movements as at least one predefined feature of the plurality of predefined features, and generating, based on the labeled baseline feature levels, the motion data.

Embodiment 18: The embodiment as in the embodiments 15-17 wherein determining, based on the motion data, the plurality of features for the predictive model comprises: determining, from the motion data, features present in two or more of the plurality of different movement data sets as a first set of candidate movements, determining, from the motion data, features of the first set of candidate movements that satisfy a first threshold value as a second set of candidate movements, and determining, from the motion data, features of the second set of candidate movements that satisfy a second threshold value as a third set of candidate movements, wherein the plurality of features comprises the third set of candidate movements.

Embodiment 19: The embodiment as in the embodiments 15-18 wherein determining, based on the motion data, the plurality of features for the predictive model comprises: determining, for the third set of candidate movements, a feature score for each of the plurality of movements associated with the third set of candidate movements, determining, based on the feature score, a fourth set of candidate movements, wherein the plurality of features comprises the fourth set of candidate movements.

Embodiment 20: The embodiment as in the embodiments 11-19 wherein training, based on the first portion of the motion data, the predictive model according to the plurality of features results in determining a feature signature indicative of at least one of predefined feature of the plurality of predefined features.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method comprising:
determining motion data associated with a plurality of movements, wherein the plurality of movements include one or more series of movements, wherein each series of movements of the plurality of movements is labeled according to a predefined feature of a plurality of predefined features;
determining, based on the motion data, a plurality of features for a predictive model;
training, based on a first portion of the motion data, the predictive model according to the plurality of features;
testing, based on a second portion of the motion data, the predictive model; and
outputting, based on the testing, the predictive model.

2. The method of claim 1, wherein determining the motion data associated with the plurality of movements comprises retrieving the motion data from a public data source.

3. The method of claim 1, wherein the plurality of movements comprise one or more of a walking set, a balancing set, a reflex set, or a motor speed set.

4. The method of claim 1, wherein determining the motion data associated with the plurality of movements comprises:
determining, based on the plurality of movements, one or more movement data sets that comprise at least one movement of the plurality of movements; and
generating, based on the one or more movement data sets, the motion data.

5. The method of claim 1, wherein the motion data is comprised of movement data from a plurality of different movement data sets.

6. The method of claim 1, wherein determining the motion data associated with the plurality of movements comprises:
determining baseline feature levels for each series of movements of the plurality of movements;
labeling the baseline feature levels for each series of movements of the plurality of movements as at least one predefined feature of the plurality of predefined features; and
generating, based on the labeled baseline feature levels, the motion data.

7. The method of claim 5, wherein determining, based on the motion data, the plurality of features for the predictive model comprises:
determining, from the motion data, features present in two or more of the plurality of different movement data sets as a first set of candidate movements;
determining, from the motion data, features of the first set of candidate movements that satisfy a first threshold value as a second set of candidate movements; and
determining, from the motion data, features of the second set of candidate movements that satisfy a second threshold value as a third set of candidate movements,
wherein the plurality of features comprises the third set of candidate movements.

8. The method of claim 7, wherein determining, based on the motion data, the plurality of features for the predictive model comprises:
determining, for the third set of candidate movements, a feature score for each of the plurality of movements associated with the third set of candidate movements; and
determining, based on the feature score, a fourth set of candidate movements,
wherein the plurality of features comprises the fourth set of candidate movements.

9. The method of claim 1, wherein training, based on the first portion of the motion data, the predictive model according to the plurality of features results in determining a feature signature indicative of at least one predefined feature of the plurality of predefined features.

10. The method of claim 1, wherein the plurality of features include at least one neurological assessment of one or more of motor function, balancing, reflex movement, sensory function, coordination, or gait.

11. A method comprising:
receiving baseline feature data associated with a plurality of movements of a subject, wherein the plurality of movements are determined from a plurality of observed movements;
providing, to a predictive model, the baseline feature data; and
determining, based on the predictive model, a neurological assessment of the subject.

12. The method of claim 11, wherein the neurological assessment comprises at least one of motor function, balancing, reflex movement, sensory function, coordination, or gait

13. The method of claim 11, further comprising training the predictive model.

14. The method of claim 13, wherein training the predictive model comprises:
determining motion data associated with the plurality of movements, wherein the plurality of movements include one or more series of movements, wherein each series of movements of the plurality of movements is labeled according to a predefined feature of a plurality of predefined features;
determining, based on the motion data, a plurality of features for the predictive model;
training, based on a first portion of the motion data, the predictive model according to the plurality of features;
testing, based on a second portion of the motion data, the predictive model; and
outputting, based on the testing, the predictive model.

15. The method of claim 14, wherein determining the motion data associated with the plurality of movements comprises:
determining, based on the plurality of movements, one or more movement data sets that comprise at least one movement of the plurality of movements; and
generating, based on the one or more movement data sets, the motion data.

16. The method of claim 14, wherein the motion data is comprised of movement data from a plurality of different movement data sets.

17. The method of claim 14, wherein determining the motion data associated with the plurality of movements comprises:
determining baseline feature levels for each series of movements of the plurality of movements;
labeling the baseline feature levels for each series of movements of the plurality of movements as at least one predefined feature of the plurality of predefined features; and
generating, based on the labeled baseline feature levels, the motion data.

18. The method of claim 16, wherein determining, based on the motion data, the plurality of features for the predictive model comprises:
determining, from the motion data, features present in two or more of the plurality of different movement data sets as a first set of candidate movements;
determining, from the motion data, features of the first set of candidate movements that satisfy a first threshold value as a second set of candidate movements; and
determining, from the motion data, features of the second set of candidate movements that satisfy a second threshold value as a third set of candidate movements,
wherein the plurality of features comprises the third set of candidate movements.

19. The method of claim 18, wherein determining, based on the motion data, the plurality of features for the predictive model comprises:
determining, for the third set of candidate movements, a feature score for each of the plurality of movements associated with the third set of candidate movements; and
determining, based on the feature score, a fourth set of candidate movements,
wherein the plurality of features comprises the fourth set of candidate movements.

20. The method of claim 14, wherein training, based on the first portion of the motion data, the predictive model according to the plurality of features results in determining a feature signature indicative of at least one of predefined feature of the plurality of predefined features.
